(19) United States
(12) Patent Application Publication
(10) Pub. No.: US 2011/0104737 A1
(43) Pub. Date: May 5, 2011

(54) Photometric measuring method for a sample liquid, a photometric measuring device, and a mixing container for a photometric measuring device

(76) Inventor: Gerhard Bonecker, Zug (CH)

(21) Appl. No.: 12/926,128

(22) Filed: Oct. 27, 2010

Related U.S. Application Data
(62) Division of application No. 12/084,695, filed on Jul. 7, 2008.

(30) Foreign Application Priority Data
Nov. 8, 2005 (AT) A 1822/2005

(51) Int. Cl. C12Q 1/02; G01N 33/50

(52) U.S. Cl. 435/29; 436/90

(57) ABSTRACT

The invention relates to a mixing container (1) for a photometric measuring device, comprising a closing element (2) that can be removed from a filling hole (3) and a first liquid (5) which is located in the interior (4) of the mixing container (1), with the mixing container (1) comprising a dosing element (8) which can be placed on the filling hole (3) of the mixing container (1) after removing the closing element (2) and adding a sample liquid, and with the dosing element (8) containing a second liquid (13) in a closed hollow space (9). In accordance with the invention, the hollow space (9) in the dosing element (8) is closed by a movable plug (10) which is discharged into the interior of the mixing container (1) along with the second liquid (13) once the second liquid (13) has been subjected to pressurization.

The invention relates to a mixing container for a photometric measuring device, comprising a closing element that can be removed from a filling hole and a first liquid which is located in the interior of the mixing container, with the mixing container comprising a dosing element which can be placed on the filling hole of the mixing container after removing the closing element and adding a sample liquid and with the dosing element containing a second liquid in a closed hollow space. The invention further relates to a photometric measuring method for a sample liquid which is mixed with a first and second liquid, with the first liquid being present in a sealed mixing container and the second liquid being present in a dosing element, as well as to photometric measuring device for performing the photometric measuring method.

In many medical tests, the sample to be measured needs to be brought into contact at first with a first liquid in order to condition the sample, prepare the same for the measurement or initiate a first chemical or biological reaction. In a second step, the second liquid is added in order to transfer the analyte of the sample to be determined to a state suitable for photometric measurement or to initiate a second chemical or biological reaction. For example, in a so-called CRP measurement (C-reactive protein) which is used for distinguishing viral or bacterial inflammations, a blood sample is mixed with a lysis reagent and thereafter a latex reagent is added and mixed, with the chemical reaction being measured with the help of a photometer.

In connection with substantially automated photometric measurements, complex automatic analyzers have become known which receive a plurality of sample cells in a sample carousel, with further a reagent carousel being provided with respective reagent agents. With the help of a pipetting device which comprises a swiveling arm, predetermined reagents can be dosed to the sample. Complex sequences of movements of the individual components such as sample carousel, reagent carousel and pipetting device need to be controlled with the help of stepper motors. The result of the reaction after the mixing of the sample with the reagents is mostly measured in a separate measuring station in a photometric way. Such systems are known for example from U.S. Pat. No. 4,965,049 A and from WO 93/20450 A1. A disadvantage of these apparatuses is the complex liquid handling because different sample and reagent liquids need to be transported from their storage and receiving containers by means of tubes and pipetting devices. It is also necessary to use automatic washing and cleaning systems in order to prevent any displacement of the sample and reagent liquid in the device.

A sample-taking and measuring element is known from WO 2005/071388 A1, which consists of several cylindrical compartments which are inserted into each other in an axially displaceable way, with their inside spaces being sealed in the initial position by a penetrable membrane. Two of the elements contain reagents and a sample can be introduced in the third element. The compartments are slid into each other by exerting pressure on the two outer elements, as a result of which the membranes tear on the connecting points and simultaneously the two reagent liquids are mixed with the sample. Analysis occurs either by optical inspection or by using a measuring device.

A similar collecting and mixing container in which the sample can also be measured is known from WO 95/25948, with the sample being obtained by a sampling swab and being introduced into a cylindrical container with several compartments. The individual compartments are sealed by membranes which are penetrated with the help of an insertion element for the sampling swab, so that the reagents are able to come into contact with the sample in the swab.

DE 24 41 724 A1 describes an analytic cartridge for photospectrometric measurements, comprising a first container for receiving a first liquid, with the container being sealed at first by a closing element. After the removal of the closing element, the sample to be analyzed is placed in the container and a container insert is then placed on the same which comprises a reagent liquid in an auxiliary chamber. The auxiliary chamber is provided with a cylindrical tappet which in the initial position protrudes beyond the container insert and which, when pressed down, tears open a membrane of the auxiliary chamber with the help of a cutting edge on the front side and thus releases the second liquid from the auxiliary chamber into the container with the first liquid. Once the liquids have dissolved and mixed completely, the container is heated in the manner required for the analytic method and the sample is measured in a photometric way.

A sample vessel for photometry is known from U.S. Pat. No. 6,495,373 B1, consisting of a container for receiving a first liquid and a shutter add-on for receiving a second liquid. The second liquid in the shutter add-on is separated by a membrane from the first liquid, with a rod-like actuating element for penetrating the membrane being provided. The actuating element acts with its tip directly on the membrane. When it is penetrated, partial pieces remain on the shutter add-on and act as a splash collector. The actuating element protrudes from the shutter add-on before its actuating, so that an erroneous actuating outside of the analytical device is possible in a disadvantageous manner. The sample containers are inserted in an analytical apparatus, with a cylinder being provided which is rotatable about a vertical axis and comprises receiving openings for the sample vessels. When a lid of the analytical apparatus is closed, it acts on the actuating element so that the membrane is penetrated.

It is the object of the invention to provide a photometric measuring method for a sample liquid which offers simplified handling, which is mixed with a first and second liquid prior to the actual measurement, and a mixing container suitable for this purpose, with any handling of liquid outside of the mixing container being avoided. Moreover, a precise dosing of the first and second liquid should be possible in a simple way, with the individual analytic steps being performable in a new improved analytical apparatus in a substantially automated way.

This object is achieved in accordance with the invention in a mixing container which already contains the first liquid in a precise dosing in such a way that the hollow space in the dosing element is closed by a movable plug which is discharged into the interior of the mixing container along with the second liquid once the second liquid has been subjected to pressurization. In contrast to the embodiment according to DE 24 41 724 A, a plug is provided as a closing element for the hollow space in the dosing element which after pressurization of the second liquid discharges into the interior of the mixing container together with the same. This leads to the consequence that no membrane parts will adhere at the discharge-side end of the dosing element to which residues of the second liquid will accumulate and thus lead to imprecision in the mixing ratio and the subsequent measurement.

In accordance with the invention, the hollow space comprises on the side opposite of the plug a displaceable sealing plunger which is arranged in the hollow space. In the invention, the axially displaceable sealing plunger which can be actuated by a stamp of the analyzer is entirely situated in the hollow space of the dosing element. In contrast to DE 24 41 724 A, the plunger in the dosing element does not act directly on the closing element, but via pressure in the liquid or the air cushion situated above the same and thus also does not protrude beyond the dosing element, so that an erroneous actuation of the apparatus prior to the use in an analyzer is excluded.

According to an especially advantageous further development of the invention, the liquid level in the mixing container is dimensioned in such a way that, at least after mixing with the second liquid, the dosing element will immerse with its discharge opening into the first liquid in the mixing container. This leads to a complete discharge (without any adhering drops) of the dosing element, thus enabling a precise mixing ratio and also a precise measuring result.

The photometric measuring method in accordance with the invention is characterized by the following steps:
Opening of the mixing container which contains the first liquid;
Adding the sample liquid;
Sealing the mixing container with a dosing element which contains the second liquid in a hollow space sealed with a plug;
Mixing of the first liquid with the sample liquid;
Introduction of the second liquid from the dosing element into the mixing container, with pressure being exerted on the second liquid and the same being discharged with the plug to the first liquid;
Mixing of the first liquid, the sample liquid and the second liquid;
Photometric measurement of the chemical reaction in an analyzer, and
Calculating the concentration of at least one sample ingredient.

A photometric calibrating measurement can be performed in accordance with the invention for obtaining an initial value for the determination of the concentration after the mixing of the first liquid with the sample liquid.

The second liquid is preferably transferred with the help of a plunger from the dosing element to the interior of the mixing container and mixed there by means of a magnetic stirring rod or ferromagnetic element (e.g. a steel ball) which is present in the mixing container.

The two elements, i.e. the mixing container with the first liquid and the dosing element with the second liquid, can be offered in a set in a sterile packaging as disposable elements and be disposed after use with the sample, with any liquid management thus becoming obsolete directly in the analyzer or outside of the mixing container.

In accordance with the invention, an information carrier such as an RFID chip or a barcode which is readable in a contactless way is arranged on the mixing container. The information carrier can also be arranged on a packaging of the mixing container and the dosing element or a packaging unit for several test sets, or be enclosed with the packaging.

In accordance with the invention, the plug consists of a material which will float in the first liquid in the mixing container, so that the photometric measurement in the liquid is not obstructed.

A photometric measuring device in accordance with the invention for measuring a sample liquid which receives in a housing a carrier unit for at least one mixing container plus inserted dosing element, with the mixing container comprising a first liquid and the sample liquid and the dosing element comprising a second liquid, is characterized in that the carrier unit is integrated in a flap which can be flipped out of the housing or an extractable drawer and is pivotable or displaceable with the same from a measuring position in the analyzer to a loading position for the mixing container.

The invention is now explained below in closer detail by reference to partly schematic drawings, wherein the drawings show the following in a sectional view:

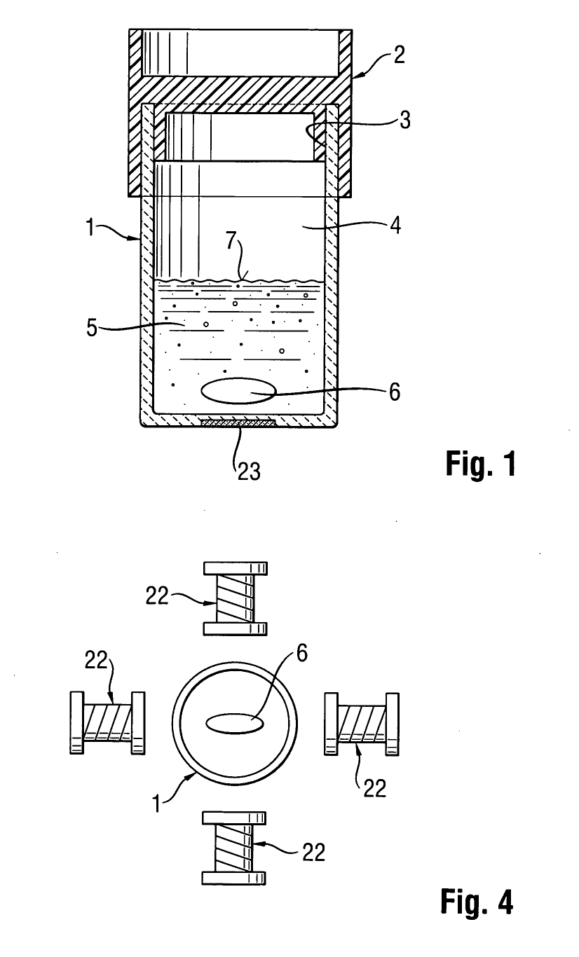

FIG. 1 shows the mixing container in accordance with the invention in the initial state;

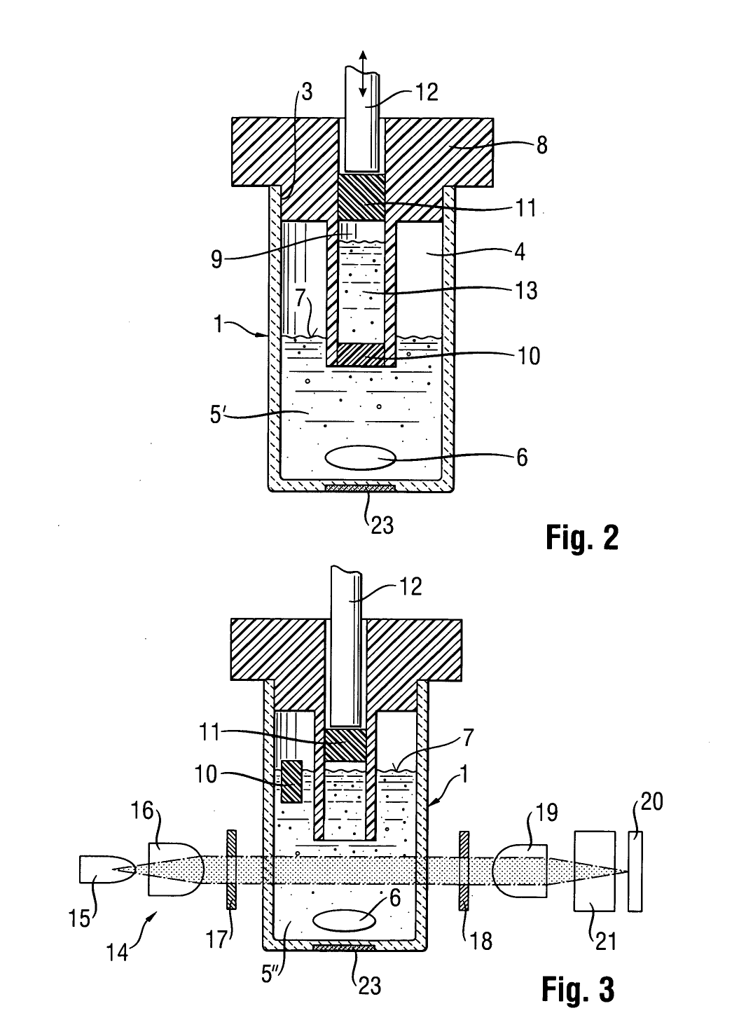

FIG. 2 shows the mixing container according to FIG. 1 with added dosing element;

FIG. 3 shows the mixing container according to FIG. 2 inserted in a photometric measuring device;

FIG. 4 shows a top view of the mixing container with a coil arrangement for the mixing unit;

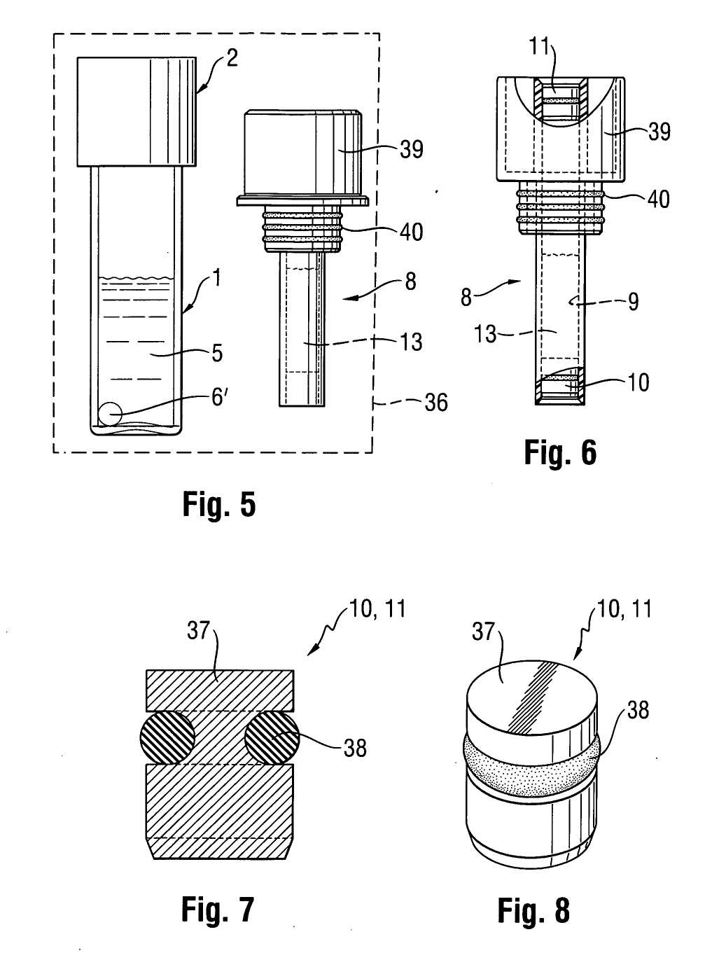

FIG. 5 shows a packaging unit consisting of a mixing container and a dosing element;

FIG. 6 shows the dosing element according to FIG. 5 in a sectional view;

FIG. 7 shows a plug of the dosing element in an enlarged sectional view;

FIG. 8 shows the plug according to FIG. 7 in a three-dimensional view;

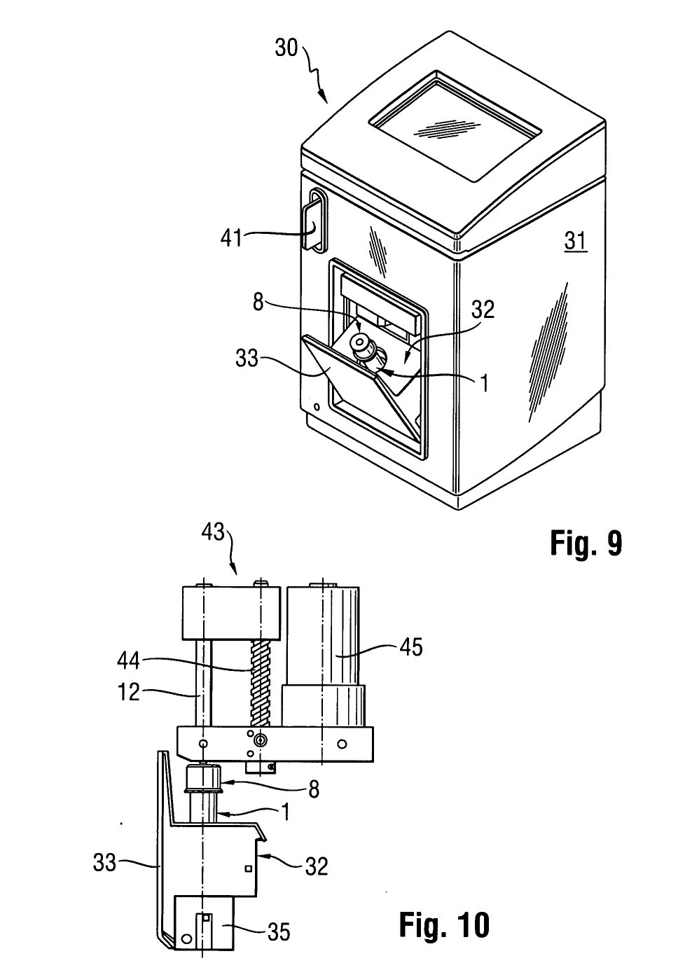

FIG. 9 shows a three-dimensional view of a photometric measuring device in accordance with the invention for performing the photometric measuring method;

FIG. 10 shows a side view of the hinged flap of the measuring device according to FIG. 9 plus carrier unit for the mixing container and actuating device for the closing plunger;

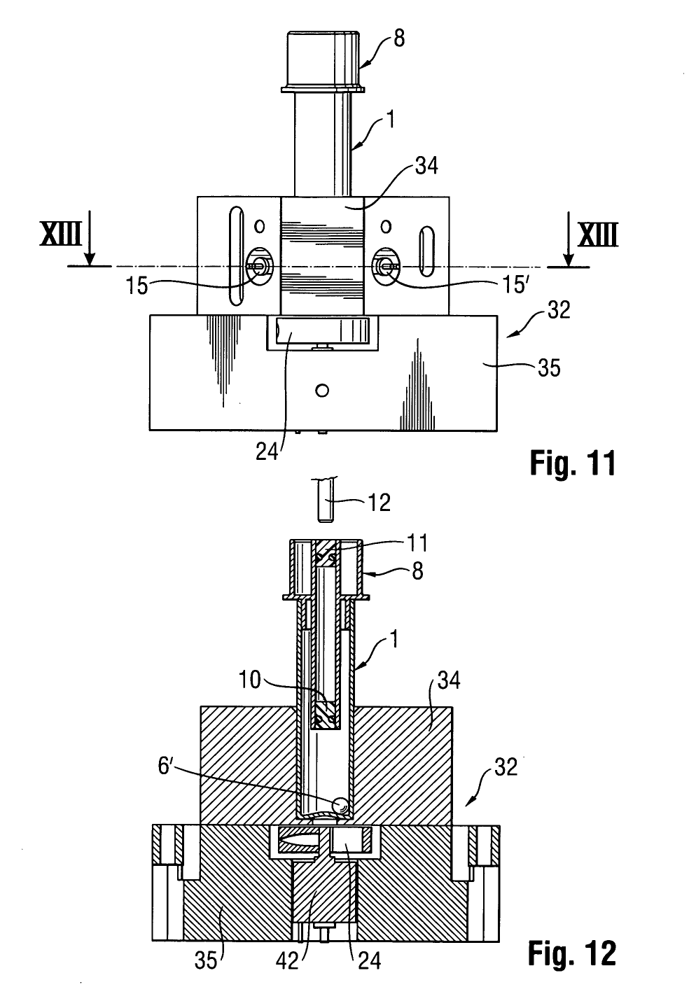

FIG. 11 shows the carrier unit according to FIG. 10 without flaps in a front view;

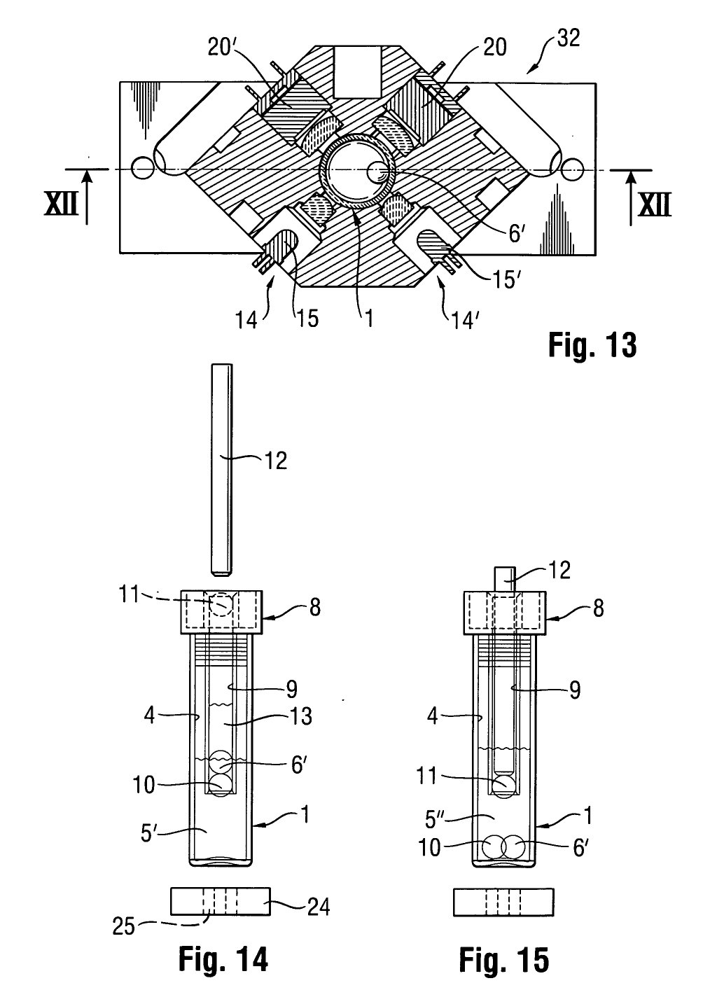

FIG. 12 shows a longitudinal sectional view through the carrier unit according to line XII-XII in FIG. 13;

FIG. 13 shows a sectional view through the carrier unit according to line XIII-XIII in FIG. 11, and

FIG. 14 and FIG. 15 show a further embodiment of the mixing container in accordance with the invention in two operating states.

The mixing container 1 as shown in FIGS. 1 to 4 is used for use in a photometric analyzer (see FIG. 9) and comprises a closing element 2, e.g. a removable plastic plug, which seals the filling hole 3. A first liquid 5 is located in the interior 4 of the mixing container 1, and a steel ball or magnetic stirring rod 6. An air space is located above the liquid 5, with the liquid surface being designated with reference numeral 7.

In order to perform a photometric measurement, the closing element 2 is removed from the mixing container 1, e.g. a measuring cell which is transparent for the measuring radiation, and a sample liquid is added with a pipette for example, so that a mixture 5′ of first liquid and sample liquid is located in the mixing container 1. Thereafter, the mixing container 1 is sealed with a dosing element 8 which seals the filling hole 3 in the same manner as the original closing element 2.

A hollow space 9 is located in the dosing element 8 which is preferably arranged in a cylindrical way and is sealed with a plug 10 (e.g. silicone plug) in the direction towards the mixture 5′. On the other side, an axially displaceable closing plunger 11 is situated in the hollow space 9, on which pressure can be exerted with a stamp 12 of the analyzer. The mixing container 1 is now inserted in the analyzer (see FIG. 9) and the magnetic stirring rod 6 is made to move in order to mix the first liquid with the sample liquid. Optionally, a first measurement is performed in order to obtain an initial value for the following concentration measurement.

The closing plunger 11 is thereafter moved downwardly with the help of stamp 12, with the occurring pressure being transmitted via an air cushion to the second liquid 13 in the dosing element 8. The plug 10 moves from the hollow space 9 as a result of the increasing pressure, so that the second liquid 13 is discharged to the mixing container 1 and a mixture 5″ is obtained.

The plug 10 consists of a material which floats in the liquid in the mixing container 1 (see FIG. 3), so that the photometric measurement is not obstructed. The liquid level 7 in the mixing container 1 is dimensioned in such a way that at least after the mixing with the second liquid 13 the dosing element 8 immerses with its discharge opening into the liquid in mixing container 1. The complete discharging (without any adhering drops) of the dosing element 8 is thus enabled.

This is followed by a further mixing process with the magnet stirring rod 6, whereupon the sample (as shown in FIG. 3) is measured with the help of a photometric device 14. It comprises a light source 15 for example such as an LED, an entrance lens 17 as well as an entrance diaphragm 17 on the input side and an exit diaphragm 18, an exit lens 19 and a photodiode 20 on the outlet side. A filter 21 can be arranged between exit lens 19 and the photodiode 20.

FIG. 4 shows an arrangement of magnet coils 22 which make the magnet stirring rod 6 arranged in the mixing container 1 move in the known manner. As an alternative it is also possible to use a motor which makes a disk (see magnetic stirring disk 24 in FIGS. 5 and 6, and FIGS. 11 and 12) rotate which is arranged beneath the mixing container, or on which or in which at least one permanent magnet 25 is arranged.

A contactless readable information carrier 23 such as an RFID chip or a barcode can be arranged on the mixing container 1 for identifying the samples. It is also possible to use one RFID chip per packaging unit with 25 or 50 tests for example. The RFID chip can contain the type, number and calibration data and expiration date of the tests, thus ensuring automation in the test recognition and increased security in making the findings.

FIG. 5 shows a test set with a mixing container 1 plus closing element 2 which is filled with the first liquid 5 and a dosing element 8 filled with a second liquid 13 in a packaging 36. The dosing element 8 comprises a handle element 39 for better handling and attached sealing rings 40 for sealing after the insertion of the mixing container 1.

As is indicated in a sectional view according to FIG. 6 with the broken line, the continuous cylindrical hollow space 9 of the dosing element 8 is sealed at the bottom end with a movable plug 10 and at the opposite end with a movable closing plunger 11. The plug 10 and the closing plunger 11 can be arranged in a similar way and, as shown in FIGS. 7 and 8, comprise a cylindrical body 37 made of an injection-moulded part which receives an O-ring 38 in a circumferential groove 4, which ring rests in a sealing manner on the cylindrical hollow space 9 of the dosing element 8.

FIG. 9 shows a photometric measuring device 30 in accordance with the invention for measuring a sample liquid, which device receives in a housing 31 a carrier unit 32 for at least one mixing container 1 plus added dosing element 8, with the mixing container 1 (as already explained above) containing a first liquid and the sample liquid and the dosing element 8 containing a second liquid. The carrier unit 32 is integrated in a flap 33 which can be swiveled out of the housing 31 and can be swiveled with the same from a measuring position in the analyzer to a loading position (see shown position) for entering the mixing container 1. It would also be possible to integrate the carrier unit 32 in a drawer which can be withdrawn from the analyzer and which is displaceable from a measuring position in the analyzer to a loading position for the mixing container 1. An actuating element of the measuring device 30 such as a stamp 13 acts in the measuring position on the closing plunger 11 (see FIG. 10 or FIG. 12) until the plug 10 and the second liquid exits from the dosing element 8 to the mixing container 1.

FIG. 10 shows the pivotable flap 33 of the measuring device plus carrier unit 32 for the mixing container 1 and an actuating device 43 for the stamp 12 in a side view. The stamp 12 is moved up and down with the help of spindle 44 which is driven by a motor 45.

As is shown in the following FIGS. 11 to FIG. 13, the carrier unit 32 comprises a receiving block 34 for the mixing container 1 which is preferably capable of thermostatting and in which two photometric devices 14, 14′ are arranged which each comprise a light source 15, 15′ and a photodiode 20, 20′, with the optical axes of the photometric devices 14, 14′ being substantially perpendicular to the longitudinal axis of the mixing container 1. Furthermore, the carrier unit 32 comprises a mixing unit 35 with a magnetic stirring disk 24 which is driven by a motor 42 and which acts upon a magnetic stirring rod or a ferromagnetic element such as a steel ball 6′ in the interior of the mixing container 1.

The two photometric devices 14, 14′ are equipped for example with two LEDs of different wavelength, with the device automatically choosing the correct test software on the basis of the data stored on the RFID chip or a data card 41 insertable into the device (see FIG. 9).

In the embodiment of a mixing container plus dosing element as shown in FIGS. 14 and 15, the sample has already been added in FIG. 14 and the dosing element 8 has already been placed on the mixing container 1. In accordance with the invention, the closing plunger 11 and the plug 10 of the dosing element 8 are each arranged as plastic balls which enclose the second liquid 13 and a steel ball 6′. The plastic balls consist of polyoxymethylene (POM or polyacetal) which seal the hollow space 9 on both sides. The ball 11 is now pressed into the same by the stamp 12 of the analyzer, as a result of which the ball forming the plug 10 and the steel ball 6′ exit to the mixing container 1 and release the second liquid. The steel ball 6′ is made to move by the magnetic stirring disk 24 with the permanent magnet 25 for mixing the liquids. It is also possible to use the steel ball 6′ as the plug 10, through which the plastic ball at the discharge opening of the dosing element 8 can be omitted.

### First Example of CRP Measurement

A measuring sequence of a CRP measurement (C-reactive protein, which is used mainly for differing between viral and bacterial inflammation) is illustrated as a first example:
Mixing container 1 is sealed with closing element 2 verschlossen and already filled with lysis reagent (1000 μl) and with a stirring part (magnetic rod or ball 6′);
Closing element 2 is removed and sample liquid (5 μl of whole blood) is added (manually, with pipette);
Mixing container 1 is sealed with dosing element 8 (contains latex reagent) and inserted in photometric measuring device 30;
Test identification by the measuring device (by RFID chip in the packaging or on the mixing container);
Lysis reagent and sample liquid are mixed with the mixing unit 35;
The calibration value is measured (optional);
Latex reagent (250 μl) is dosed and added with the help of the stamp 12 of the photometric measuring device 30;
Lysis reagent, sample liquid (whole blood) and latex reagent are mixed with the help of the mixing unit 35;
The chemical reaction is measured with the help of the photometer;
The concentration is determined.

The measuring range of the photometric measuring device is 0.2 to 6 mg/dl for example.

### Second Example

### HbA1c Test

The Hba1c value, which is generally also known as “blood sugar memory”, allows drawing conclusions on the blood sugar level. The sugar haemoglobin HbA1c is measured in this method in a blood sample (lyzed whole blood). It is examined as to how much blood pigment (haemoglobin) is bonded with sugar (glucosed).

The determined HbA1c value shows the amount of the average blood sugar values during the last six to twelve weeks. The normal value is lab-dependent and lies close to four to six percent (standard 4 to 6%). The percentage value stands for the share of the glucosed haemoglobin in comparison with total haemoglobin.

The first HbA1c reagent is located in the mixing container. The second HbA1c reagent is in the dosing element. Test sequence occurs as in example 1.

The object of diabetes therapy is the decrease of HbA1c beneath 6.5 percent.

### Third Example

### HCY Test

From a chemical standpoint, homocystein (HCY) belongs to the group of the so-called amino acids. In the body, homocystein is formed from methionine, another amino acid, which is supplied with food. Homocystein is normally degraded very rapidly, with vitamin B6 (pyridoxine), vitamin B12 (cobalamin) and folic acid being required.

Homocystein was identified as a separate risk factor for atherosclerotic or thromboembolic events (peripheral arterial occlusive vascular disease, stroke, coronary heart disease (angina, cardiac infarction), occlusive changes to the carotid artery). In a number of further diseases such as old-age dementia, development of defects in the neural canal (spina bifida) of the child in the womb and anemia, a connection with increased homocystein levels was established.

The first HCY reagent is located in the mixing container. The second HCY reagent is located in the dosing element. The test sequence occurs as in example 1.

Target range for homocystein is below 10 μmol/l in the serum.

1-9. (canceled)

10. A photometric measuring method for determining at least one sample ingredient of a sample liquid which is mixed with a first and a second liquid, with the first liquid being present in a sealed mixing container and the second liquid in a dosing element, comprising the following steps:
opening the mixing container containing the first liquid;
adding the sample liquid to the first liquid in the mixing container;
sealing the mixing container with a dosing element, which contains the second liquid in a hollow space sealed with a plug;
mixing of the first liquid with the sample liquid;
applying pressure on the second liquid so that the second liquid and the plug are discharged from the hollow space of the dosing element into the first liquid in the mixing container;
mixing of first liquid, the sample liquid and the second liquid in the mixing container to create a chemically reacted mixture;
photometrically measuring the chemically reacted mixture in an analyzer, and
calculating a concentration of at least one sample ingredient in the chemically reacted mixture.

11. The method according to claim 10, including photometrically calibrating the mixed first liquid and sample liquid.

12. The method according to claim 10, wherein the mixing of the sample liquid with the first liquid and the second liquid is accomplished using a magnetic stirring rod or ferromagnetic element present in the mixing container.

13. The method according to claim 10, wherein the second liquid is discharged from the hollow space of the dosing element to the interior of the mixing container using a plunger.

14-17. (canceled)
